(19) United States
(12) Patent Application Publication
(10) Pub. No.: US 2009/0042183 A1
(43) Pub. Date: Feb. 12, 2009

(54) Use of pcr-based techniques to analyze compositions of botanicals

(76) Inventors: Ann M. Hirsch, Santa Monica, CA (US); Michelle R. Lum, Los Angeles, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/658,623

(22) PCT Filed: Jul. 14, 2005
(86) PCT No.: PCT/US05/25320

§ 371 Date: Sep. 18, 2008

Related U.S. Application Data
(60) Provisional application No. 60/592,228, filed on Jul. 28, 2004.

(51) Int. Cl. C12Q 1/68

(52) U.S. Cl. 435/6

(57) ABSTRACT

Methods for use in identifying the individual biological components present in a botanical mixture are provided. Using a combination of genomic-locus specific PCR, single strand conformation polymorphism, and sequence analysis, the biologic components of a botanical composition are identified without prior knowledge as to which components may be present.

### BACKGROUND OF THE INVENTION

According to the World Health Organization, ˜4 billion people worldwide use botanical remedies as all or part of their primary health care needs. Many of these people reside in developing countries, but more and more people in the developed world have turned to the use of botanicals for health enhancement. The number of botanical species that have been exploited for their potential health benefits is enormous and includes both staple food sources such as fruits, vegetables, and grains, as well as herbs used in traditional medicines. In the United States alone, over 80 million people use some type of herbal or botanical dietary supplement.

However, even as more information is obtained on the bioactivity of the phytochemicals compounds in dietary supplements, the efficacy of botanical supplements used to deliver them is questionable due to lack of standardization and quality standards in cultivation and processing of the plant material. Analysis of different commercial products has found them to be highly variable in pharmacologically relevant substances (Osowski et al., 2000; Kressmann et al., 2002). Such variability could be due to a multitude of factors, including different harvesting and processing techniques used by different manufacturers. One concern is the authenticity of the botanical supplements themselves, which is highlighted by actual incidents where the presence of undesirable botanicals resulted in illness. In one case, two women were hospitalized because of digoxin poisoning after-taking an herbal product that was contaminated with Digitalis lanata (Slifman et al., 1998). In another instance, a number of patients in a clinical weight loss trial developed severe nephrotoxicity, in some cases resulting in irreversible damage and the need for kidney transplants (Vanherweghem et al., 1993). This was determined to be due to the presence of Aristolochiza fangchi, which had been supplied instead of Stephania tetrandra (Vanherweghem et al., 1998). The Dietary Supplement Health Education Act of 1994 mandates the development of good manufacturing practices (GMPs) as one approach to solving this problem.

Botanicals in dietary supplements are sold in many forms, including extracts and dried plant material. The latter usually consists of dried and fragmented leaves, roots, or flowers sold in capsules or pills. Traditionally, identification of the composition of these types of plant material has been done by direct observation of the morphological characteristics of the preparation, including leaf or stem trichomes, or various histological features, e.g., distinctive cell types. This type of analysis can often identify the biological components to the genus level and sometimes to the species level. However, these methods are frequently inaccurate, especially for plant material that is either ground to a very fine powder, field-collected and contaminated with other plants, or highly oxidized or mechanically degraded due to drying or storage under unfavorable conditions.

Alternative methods for the authentication of botanical products include a variety of molecular biology techniques that focus on confirming and/or distinguishing between different species. The polymerase chain reaction (PCR) has been applied at various levels in many of these assays. PCR products generated from botanical compositions have been directly sequenced to identify particular species. In addition, PCR followed by Restriction Fragment Length Polymorphism (RFLP), Random Amplified Polymorphic DNA (RAPD), and Amplified Fragment Length Polymorphism (AFLP) have been used to generate profiles for specific species in a botanical sample or distinguish between specific species. As employed thus far, however, these methods rely on having prior knowledge of the species expected to be present in the botanical mixture so that the reagents used to produce the molecular profile are appropriately designed and used.

One significant problem in performing many of the molecular biology techniques for species identification is obtaining PCR-competent material from botanical dietary supplements or other botanical sources, because the DNA is often degraded or fragmented due to harsh conditions during processing or storage.

In view of the widespread use of botanicals, and importance of correct identification, improved and efficient methods of identification of the primary and contaminating botanicals are of particular interest. The present invention addresses this need.

### Relevant Literature

Leroy et al. (2002) “Characterization and identification of alfalfa and red clover dietary supplements using a PCR-based method” J. Agric. Food Chem. 50: 5063-5069; Lopez et al. (2002) “Characterization of genetic markers for in vitro cell line identification of the marine sponge Axinella corrugate.” J. Heredity. 93:27-36; Schwieger et al. (1998) “A new approach to utilize PCR-single-strand-conformation polymorphism for 16s rRNA gene-based microbial community analysis.” Applied and Environ. Microbiol. 64: 4870-4876; Cheng et al. (2000) “RAPD analysis of Astragalus medicines marketed in Taiwan” American Journal of Chinese Medicine. 28: 273-278; Kohjyouma et al. (2000) “Intraspecific variaton in Cannabis sativa L. Based on intergenic spacer region of chloroplast DNA” Biological and Pharmaceutical Bulletin. 23: 727-730; Orita et al. (1989) “Detection of polymorphisms of human DNA by gel electrophoresis as single-strand conformation polymorphisms.” Proceedings of the National Academy of Science USA. 86:2766-2770; Shaw et al. (2002) “Authentication of Chinese Medicinal Materials by DNA Technology” World Scientific Publishing Co. Pte. Ltd. Singapore, pg. 11-12, 73-74; Pusch et al. (1998) Nucleic Acids Research 26:857-9; Schilter et al. (2003) Food Chemistry and Toxicology 41:162549; Calixto et al. (2000) Brazilian Journal of Medical and Biological Research 33:179-89; Techen et al. (2004) Current Medicinal Chemistry 11:1391-1401; Pierson et al. (2004) Current Medicinal Chemistry 11:1361-74; Walker (2004) Toxicology Letters 149:187-95; Blattner (1999) “Direct amplification of the entire ITS region from poorly preserved plant material using recombinant PCR” Biotechniques 27:1180-1186; Brisken et al. (2000) “Influence of nitrogen on the production of hypericins by St. John's wort” Plant Physiol. Biochem. 38:1-8; Doyle et al. (1997) “A phylogeny of the chloroplast gene rbcL in the Leguminosae: taxonomic correlations and insights into the evolution of nodulation” Amer. J. Bot. 84:541-554; Hayashi (1991) “PCR-SSCP: a simple and sensitive method for detection of mutations in the genomic DNA” PCR Methods and Applications 1:34-38; Jansa et al. (2002) “Intra- and intersporal diversity of ITS rDNA sequences in Glomus intraradices assessed by cloning and sequencing, and by SSCP analysis” Mycol. Res. 106: 670-681; Kressmann et al. (2002) “Pharmaceutical quality of different Ginkgo biloba brands” J. Pharm. Pharmacol. 54: 661-669; Mihalov et al. (2000) “DNA identification of commercial ginseng samples” J. Agric. Food Chem. 48: 3744-3752; Mora et al. (2003) “16S-23S rRNA intergenic spacer region sequence variation in Streptococcus thermophilus and related dairy streptococci and development of a multiplex ITS-SSCP analysis for their identification” Microbiology 149: 807-813; Osowski et al. (2000) “Pharmaceutical comparability of different therapeutic Echinacea preparations” Res. Compl. Nat. Classical Med. 7: 294-300; Slifman et al. (1998) “Contamination of botanical dietary supplements by Digitalis lanata” New England Journal of Medicine. 339: 806-811; Vanherweghem et al. (1993) “Rapidly progressive interstitial fibrosis in young women: association with slimming regimen including Chinese herbs” Lancet 341: 387-391; Vanherweghem (1998) Misuse of herbal remedies: the case of an outbreak of terminal renal failure in Belgium (Chinese herbs nephropathy)” J. Altern. Complement. Med. 4: 9-13. Kojoma et al. (2002) “Genetic identification of cinnamon (Cinnamomun spp.) based on the tmL-tmF chloroplast DNA” Planta Med. 68: 94-96.

### SUMMARY OF THE INVENTION

Methods are provided for identifying individual biological genetic components present in a botanical mixture. By providing a means to positively identify the primary and contaminating constituents present in the botanical composition, the methods disclosed herein find use in assessing the integrity of botanical compositions that are used as foods, dietary supplements, therapeutics, and the like. The methods of the invention utilize a combination of genomic-locus specific PCR, single strand conformation polymorphism (SSCP), and sequence analysis. Benefits are provided by the speed and efficiency of the methods in providing information on the biologic components of a botanical composition without requiring prior knowledge as to which botanicals may be present.

Biological species present in a food product or dietary supplement containing one or a mixture of unknown botanical species are identified. DNA isolated from a botanical composition is repaired if necessary and subjected to PCR amplification using primer pairs that are specific for a genomic identification region that is substantially homologous, but not identical, among a wide variety of botanical species. From this amplification product, single stranded DNA is generated and fractionated by electrophoresis through a matrix, e.g., acrylamide, under non-denaturing conditions. Migration of the single stranded DNA products through the matrix depends on their structural conformation, which is directly influenced by the nucleotide sequence (called single-strand conformational polymorphism or SSCP). Individual bands, representing PCR products with distinct sequences, are identified and isolated from the matrix and sequenced to positively identify the species from which it was derived. The disclosed method allows not only the confirmation of primary biologic component(s) of a composition, but also the detection and identification of unknown, or contaminating, biologic components that may be present.

### BRIEF DESCRIPTIONS OF THE FIGURES

FIGS. 1A-1C: Agarose gel electrophoresis of genomic DNA and the ITS-2 region. Genomic DNA isolated from the contents of commercial alfalfa or red clover capsules was severely degraded as indicted by a smear. B) The ITS-2 region was successfully amplified from DNA isolated from fresh tissue and from capsules of alfalfa of company B, but not from the DNA from company A. C) After a repair reaction, the ITS-2 region could be amplified from DNA from company A.

FIG. 2: Comparison of the ITS-1 and ITS-2 regions and two species of licorice using SSCP. The ITS-1 region did not distinguish between European and Chinese licorice. In contrast, the ITS-2 region showed different migration of ssDNA products.

FIG. 3: SSCP analysis can be used to distinguish between different plant species.

FIG. 4: SSCP can detect a woad “contaminant”. SSCP analysis of alfalfa containing a simulated woad contaminant showed that the woad could still be detected when present at a 1:5000 dilution.

FIG. 5: SSCP analysis of commercial alfalfa and red clover products. SSCP analysis shows that each of the commercial alfalfa and red clover products produce a PCR product that co-migrates with that of a known alfalfa or red clover sample. However, each of the commercial products contains additional, faster migrating bands.

### DEFINITIONS

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule. Thus, the terms include triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA.

Unless specifically indicated otherwise, there is no intended distinction in length between the terms “polynucleotide,” “oligonucleotide,” “nucleic acid” and “nucleic acid molecule” and these terms will be used interchangeably. These terms refer only to the primary structure of the molecule. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

As used herein the term “isolated,” when used in the context of an isolated compound, refers to a compound of interest that is in an environment different from that in which the compound naturally occurs. “Isolated” is meant to include compounds that are within samples that are substantially enriched for the compound of interest and/or in which the compound of interest is partially or substantially purified. The term “isolated” encompasses instances in which the recited material is unaccompanied by at least some of the material with which it is normally associated in its natural state, preferably constituting at least about 0.5%, more preferably at least about 5% by weight of the total protein in a given sample. For example, the term “isolated” with respect to a polynucleotide generally refers to a nucleic acid molecule devoid, in whole or part, of sequences normally associated with it in nature; or a sequence, as it exists in nature, but having heterologous sequences in association therewith; or a molecule disassociated from the chromosome.

“Purified” as used herein means that the recited material comprises at least about 75% of the total by weight with at least about 80% being preferred, and at least about 90% being particularly preferred. As used herein, the term “substantially pure” refers to a compound that is removed from its natural environment and is at least 60% free, preferably 75% free, and most preferably 90% free from other components with which it is naturally associated.

A polynucleotide “derived from” or “specific for” a designated sequence, such as a target sequence of a target nucleic acid, refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding to, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived or specific for. Polynucleotides that are “derived from” or “specific for” a designated sequence include polynucleotides that are in a sense or an antisense orientation relative to the original polynucleotide.

As used herein, the term “target nucleic acid region” or “target nucleic acid” or “target molecules” refers to a nucleic acid molecule with a “target sequence” to be detected (e.g., by amplification). The target nucleic acid may be either single-stranded or double-stranded and may or may not include other sequences besides the target sequence (e.g., the target nucleic acid may or may not include nucleic acid sequences upstream or 5′ flanking sequence, may or may not include downstream or 3′ flanking sequence, and in some embodiments may not include either upstream (5′) or downstream (3′) nucleic acid sequence relative to the target, sequence. Where detection is by amplification, these other sequences in addition to the target sequence may or may not be amplified with the target sequence.

The term “target sequence” refers to the particular nucleotide sequence of the target nucleic acid to be detected (e.g., through amplification). The target sequence may include a probe-hybridizing region contained within the target molecule with which a probe will form a stable hybrid under desired conditions. The “target sequence” may also include the complexing sequences to which the oligonucleotide primers complex and be extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands. Moreover, where sequences of a “target sequence” are provided herein, it is understood that the sequence may be either DNA or RNA. Thus where a DNA sequence is provided, the RNA sequence is also contemplated and is readily provided by substituting “T” of the DNA sequence with “U” to provide the RNA sequence.

“Homology” refers to the percent similarity between two polynucleotide or two polypeptide moieties. Two DNA, or two polypeptide sequences are “substantially homologous” to each other when the sequences exhibit at least about 50%, preferably at least about 75%, more preferably at least about 80%, at least about 85%, preferably at least about 90%, and most preferably at least about 95% or at least about 98% sequence similarity over a defined length of the molecules. As used herein, substantially homologous also refers to sequences showing complete Identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100.

Readily available computer programs can be used to aid in the analysis of homology and identity, such as LASERGENE from DNASTAR, Inc; and ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence homology are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent homology of a particular nucleotide sequence to a reference sequence can be determined using the homology algorithm of Smith and Waterman with a default scoring table and a gap penalty of six nucleotide positions.

Another method of establishing percent homology in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence homology.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by ═HIGH SCORE; Databases=non-redundant, GenBank+EMBL+DDBJ+PDB+GenBank CDS translations+Swiss protein+Spupdate+PIR. Details of these programs can be found on the internet on a website sponsored by the National Center for Biotechnology Information (NCBI) and the National Library of Medicine (see www.ncbi.nlm.gov/cgi-bin/BLAST).

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., Molecular Cloning: A Laboratory Manual, Ed. Janssen, Cold Spring Harbor Laboratory Press.

A “DNA-dependent DNA polymerase” is an enzyme that synthesizes a complementary DNA copy from a DNA template. Examples include DNA polymerase I from E. coli and bacteriophage T7 DNA polymerase. All known DNA-dependent DNA polymerases require a complementary primer to initiate synthesis. Under suitable conditions, a DNA-dependent DNA polymerase may synthesize a complementary DNA copy from an RNA template. Some DNA-dependent DNA polymerases are very heat stable and find use in the polymerase chain reaction (PCR). Examples include DNA-dependent DNA polymerase derived from Thermus aquaticus, i.e., Taq polymerase.

As used herein, a “DNA ligase” or “ligase” is an enzyme that catalyzes the formation of a phosphodiester bond between juxtaposed 5′ phosphate and 3′-hydroxyl termini in duplex DNA or RNA with blunt or cohesive-end termini. DNA Ligase can also repair single-strand nicks in duplex DNA, RNA or DNA/RNA hybrids.

As used herein, the term “lambda exonuclease” is an enzyme with a highly processive 5′ to 3′ exodeoxyribonuclease activity that selectively digests the 5′-phosphorylated strand of double-stranded DNA, exhibits greatly reduced activity on single-stranded DNA and non-phosphorylated DNA, and has no activity at nicks and limited activity at gaps in DNA.

Primers are usually single-stranded for maximum efficiency in amplification, but may alternatively be double-stranded. If double-stranded, the primer is usually first treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

A “primer pair” as used herein refers to first and second primers having nucleic acid sequence suitable for nucleic acid-based amplification of a target nucleic acid. Such primer pairs generally include a first primer having a sequence that is the same or similar to that of a first portion of a target nucleic acid, and a second primer having a sequence that is complementary to a second portion of a target nucleic acid to provide for amplification of the target nucleic acid or a fragment thereof. Reference to “first” and “second” primers herein is arbitrary, unless specifically indicated otherwise. For example, the first primer can be designed as a “forward primer” (which initiates nucleic acid synthesis from a 5′ end of the target nucleic acid) or as a “reverse primer” (which initiates nucleic acid synthesis from a 5′ end of the extension product produced from synthesis initiated from the forward primer). Likewise, the second primer can be designed as a forward primer or a reverse primer.

As used herein, the term “probe” or “oligonucleotide probe”, used interchangeably herein, refers to a structure comprised of a polynucleotide, as defined above, that contains a nucleic acid sequence complementary to a nucleic acid sequence present in the target nucleic acid analyte (e.g., a nucleic acid amplification product). The polynucleotide regions of probes may be composed of DNA, and/or RNA, and/or synthetic nucleotide analogs. Probes are generally of a length compatible with its use in specific detection of all or a portion of a target sequence of a target nucleic acid, and are usually in the range of between 8 to 100 nucleotides in length, such as 8 to 75, 10 to 74, 12 to 72, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. The typical probe is in the range of between 10-50 nucleotides long, such as 15-45, 18-40, 20-30, 21-28, 22-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

The term “assessing” includes any form of measurement, and includes determining if an element is present or not. The terms “determining”, “measuring”, “evaluating”, “assessing” and “assaying” are used interchangeably and includes quantitative and qualitative determinations. Assessing may be relative or absolute. “Assessing the presence of” includes determining the amount of something present, and/or determining whether it is present or absent. As used herein, the terms “determining,” “measuring,” and “assessing,” and “assaying” are used interchangeably and include both quantitative and qualitative determinations.

“Precision” refers to the ability of an assay to reproducibly generate the same or comparable result for a given sample.

“Accuracy” refers to the ability of an assay to correctly detect a target molecule in a blinded panel containing both positive and negative specimens.

As used herein, the term “single-strand conformational polymorphism” or “SSCP” refers to the process of identification and/or separation of single-stranded nucleic acids based on subtle differences in mobility when electrophoresed through a non-denaturing gel. Because single stranded DNA species that vary at a single nucleotide have measurable mobility differences in this assay, SSCP is most often used to analyze the polymorphisms at a single genetic locus. Like restriction fragment length polymorphisms (RFLP), SSCP are allelic variants of inherited, genetic traits that can be used as genetic markers. Unlike RFLP analysis, however, SSCP analysis can detect DNA polymorphisms and mutations at any location in single stranded DNA fragments.

A “botanical,” “botanical mixture,” or “botanical composition” is a preparation that is derived from one or a number of plants and is intended for use as a food, a dietary supplement, and/or a therapeutic. Botanicals are derived from any part of a plant (e.g., seed, root, stem, flower, or leaf) and come in a variety of forms (e.g., intact plants or plant parts, dried components, extracts, powdered preparations, capsules, ointments, etc.). Examples of commonly used plants for botanicals include, but are not limited to: Medicago sativa (alfalfa), Trifolium pratense (red clover), Glycymmiza uralensis (European licorice), Glycymmiza glabra (Chinese licorice), Isatis indigotica (woad), Aloe barbadensis (aloe vera), Echinacea angustifolia (echinacea), Eucalyptus globules (eucalyptus leaves), Linum usitatissimum (flax seed), Ginkgo biloba (ginkgo leaves), Panax quinquefolius (American ginseng root), Lavandula officinalis (lavender flowers), Podophyllum peltatum (mandrake root), Mentha piperita (peppermint leaves), Hemidesmus indicus (India sarsaparilla root), Dioscarea villosa (wild yam root), and Achillea millefolium (yarrow flowers).

As used herein, “primary” biological constituents of a botanical composition are those constituents that the composition claims to possess. For example, a dietary supplement said to be derived from alfalfa would be expected to have components derived from the plant Medicago sativa.

As used herein, “contaminating” biological constituents of a botanical composition are those constituents that the composition does not claim to possess. For example, a dietary Supplement said be derived solely from alfalfa would not be expected to have components derived from the plant Trifolium pratense (red clover) or any other plant.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DESCRIPTION OF THE SPECIFIC EMBODIMENTS

The subject invention discloses methods for use in identifying the primary and contaminating biological components present in a botanical mixture, using a combination of genomic-locus specific PCR, single strand conformation polymorphism, and sequence analysis. It is of interest to those that need to assess the integrity of botanical compositions that are used as foods, dietary supplements, or therapeutics.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Although any methods, devices and materials similar or equivalent to those described herein can be used in the practice or testing of the invention, representative methods, devices and materials are now described.

All publications mentioned herein are incorporated herein by reference for the purpose of describing and disclosing the components that are described in the publications that might be used in connection with the presently described invention.

### Methods

As summarized above, the subject invention provides methods for determining the identity of the primary and contaminating biological species in a botanical food, dietary supplement, or therapeutic. The method disclosed combines DNA purification and repair, PCR amplification of a specific genomic region, SSCP analysis, and sequence comparison to accomplish this. By botanical food, dietary supplement, or therapeutic is meant any composition containing plants, plant extracts, or more purified components thereof, alone or in combination which is intended for consumption or therapeutic use. Such compositions may also contain non plant-based components. These compositions can be in the form of pills, capsules, powders or dried preparations, balms, lotions, creams, inhalants, lozenges, liquid preparations, or any of a number of formulations intended for consumption or therapeutic use.

### DNA Isolation and Repair

In practicing the subject invention, DNA is isolated from a composition of interest containing or expected to contain one or a mixture of botanical components. Methods for isolating DNA from botanical samples are known in the art and include modified cetyltrimethylammonium bromide (CTAB) procedures or commercial kits (DNeasy Plant Mini Kit; Qiagen Inc., Valencia, Calif.).

The DNA sample is generally subjected to a repair reaction. Methods of repair are known in the art (Leroy et al., 2002; Pusch et al., 1998). In some embodiments, the repair reaction is a 2-step process comprising a nucleotide fill-in reaction followed by a DNA ligation reaction. In some of these embodiments, the fill-in reaction comprises incubating at least 10 nanograms (ng), often at least 100 ng, usually at least 1000 ng, and sometimes at least 10,000 ng of the isolated DNA with an exonuclease-free DNA polymerase, e.g., E. coli DNA polymerase 1, in an appropriate reaction buffer. The reaction mix may further contain all four deoxyribonucleotides (dNTPs). The reaction is incubated for a period of time necessary to fill in substantially all gaps and other single stranded regions, e.g., at least about 1 hour, often 5 hours, usually 1 day, and sometimes 2 days or more. The reaction is usually terminated with an appropriate stop buffer or by heat inactivation.

The ligation reaction comprises incubating the polymerase-treated DNA with a DNA ligase in an appropriate reaction buffer for a period of time required to substantially seal the nicks in the DNA. In some embodiments, a temperature cycle ligation reaction is performed in which the temperature is cycled every 10 seconds between 10° C. and 30° C. for at least, 12 hours.

The DNA may be re-purified, or “cleaned up”, prior to performing the PCR step. Methods for cleaning up a DNA sample prior to PCR are known in the art and include phenol/chloroform extraction followed by ethanol precipitation and commercially available kits (i.e. PCR Purification Kit; Qiagen Inc., Valencia, Calif.).

### PCR Amplification of a Genomic Identification Region

Once the DNA sample is prepared, PCR is performed using primer pairs that are specific for a genomic identification region. The primer pairs consist of a forward and reverse primer which can initiate synthesis of a complementary nucleic acid strand when placed under conditions in which synthesis of a primer extension product is induced, e.g., in the presence of nucleotides and a polymerization-inducing agent such as a DNA or RNA polymerase and at suitable temperature, pH, metal concentration, and salt concentration. Each primer of the primer pair is generally of a length compatible with its use in synthesis of primer extension products, and they usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. In some embodiments, the primers are usually not more than about 10, 12, 15, 20, 21, 22, 23, 24, 25, 26, 27, 28, 29, 30, 35, 40, 45, 50, 55, 60, 65, or 70 nucleotides in length.

In some embodiments, the reverse primer of the primer pair contains a 5′ terminal phosphate group. This modification allows for the production of single stranded DNA from the PCR product, which can be used for SSCP analysis (Schwieger and Tebbe, 1998).

A genomic identification region of interest is the nuclear ribosomal RNA-encoding locus containing the internal transcribed spacer (ITS) regions ITS-1 and/or ITS-2. In some embodiments, at least 10 ng, sometimes at least 100 ng, often at least 1000 ng, and up to at least 10,000 ng or more of isolated DNA is PCR amplified using a primer pair specific for the ITS-1 and/or ITS-2 region.

Generation of ssDNA and SSCP Analysis

Single stranded DNA is generated from the PCR product amplified from the desired genomic identification region. In many embodiments, the PCR product is purified prior to generation of single stranded DNA. Methods to purify PCR products are well known in the art and include commercial kits (i.e. Qiaquick PCR Purification Kit, Qiagen, Valencia, Calif.). In some embodiments, as mentioned above, the reverse PCR primer of the specific primer pair is phosphorylated at the 5′ terminus. In these embodiments, single stranded DNA may be generated from the PCR product by treating it with an exonuclease that selectively digests the phosphorylated strand of double-stranded DNA, e.g., lambda exonuclease. The exonuclease may be mixed with the PCR product in an appropriate buffer and incubated for a time to convert substantially the double stranded DNA to single stranded DNA. This incubation may be at least 1 hour, often up to at least 5 hours, sometimes up to at least 12 hours or more. The resultant single stranded DNA may be purified using standard methods known in the art, including phenol-chloroform extraction followed by ethanol precipitation or using commercially available kits (i.e. Qiaquick PCR Purification Kit, Qiagen, Valencia, Calif.), and resuspended in an amount of buffer that is compatible SSCP analysis.

In some embodiments, the single stranded DNA is fractionated by non-denaturing electrophoresis such that each DNA species may be identified and isolated based on single strand conformation polymorphism, or SSCP. Single-stranded DNA forms secondary structures based on its nucleotide composition, or sequence, with even single nucleotide differences between DNA species potentially resulting in a difference in structure. When electrophoresed through a matrix under non-denaturing conditions, these structural differences impart unique migratory properties to each of the single strand DNA species allowing one to visualize each variant. This technique has most often been applied to analyses of allelic variation at a single genetic locus. The single stranded DNA sample may be denatured prior to electrophoresis, e.g., by adding formamide and sodium hydroxide or incubating at high temperature (e.g., 95° C. for 2 minutes). The single stranded DNA sample is usually electrophoresed through an acrylamide matrix (e.g., capillary electrophoresis or slab gel electrophoresis) under non-denaturing conditions. Other matrices include MDE (FMC Bioproducts, Rockland, Me.) which is specifically formulated for SSCP analysis. The matrix length through which the single stranded DNA is electrophoresed varies and can be at least 10 cm, often at least 20 cm, usually at least 50 cm, and sometimes at least 70 cm or more.

The individual bands corresponding to the various single stranded DNA species can be visualized on the gel using a variety of methods known to those skilled in the art, e.g., ethidium bromide staining or silver staining. After staining, each distinct band detected can be excised from the gel for further processing. In some embodiments, each band is excised and transferred to a tube containing elution buffer.

The subject invention provides methods for obtaining positive identification of the biological components of a botanical composition. In some embodiments, the single strand DNA isolated from the SSCP gel is subjected to DNA sequencing. The single strand DNA can be sequenced directly. Alternatively the single strand DNA can be subjected to another round of PCR to obtain more template DNA for the sequencing reaction. In some of these embodiments, the single strand DNA is used as template in a PCR reaction with the same primer pair employed for the initial PCR amplification reaction, i.e., the reaction performed on the DNA isolated from the botanical composition. The resultant PCR product can be purified and sequenced using any of a variety of methods known to those of skill in the art.

As mentioned above, the ribosomal RNA ITS region is a genomic identification region of interest. This particular genomic region is useful because it has been sequenced in a wide variety of botanicals, with this information being readily available in public databases. Therefore, by comparing the DNA sequence obtained from the single stranded DNA species isolated using the methods of the subject invention to the sequences in public databases, one can make a strong conclusion as to the identity of the species present in the botanical composition under investigation. In instances where the exact DNA sequence obtained is not present in any known database, it may be possible to classify the organism from which the genomic identification region was amplified to the genus or even the level based on the sequence similarity to known organisms. This type of phylogenetic analysis for the classification of organisms is known to those of skill in the art.

While the ITS of ribosomal RNA is useful as a genomic identification region in practicing the subject invention, it is by no means the only genomic region suitable for this method and therefore the scope of the methods disclosed herein should not be limited to analysis of only that genomic region. For instance, the chloroplast gene rbcL (Rubisco large subunit) has also been used extensively in phylogenetic analyses and could be applied to this method.

### Utility

The subject invention finds use in identifying primary and contaminating biologic components of botanicals that are used as foods, dietary supplements, and/or therapeutics. This method does not require prior knowledge as to the specific botanical species that may be present in the botanical composition. As such, the subject invention can be used for quality control in the production and distribution of botanical compositions making their use by the public more safe and effective.

The methods disclosed herein can be employed to test the source of plant material used to generate botanical compositions for contaminants, e.g., unknown plants that were growing amongst the primary plants prior to harvesting.

As many botanical supplements and therapies are made at a single processing facility, the methods disclosed herein can be used to detect whether cross-contamination during production has occurred.

This method can also be modified to identify primary and contaminating biological components of mixtures other than botanicals by using alternative PCR primer pairs that amplify a genome identification locus from other classes of organism, e.g., bacterial ribosomal gene locus.

### Kits

Kits for use in the subject invention may also be provided. Such kits include at least a set of primer pairs for use in the amplification of the genomic identification region of interest and the reagents to generate single stranded DNA. Kits may also contain the reagents for isolating nucleic acid from the sample of interest, the reagents to perform the DNA repair reaction, and/or the reagents necessary to perform the SSCP fractionation and DNA isolation. Kits may also contain instructions for using the kit to detect primary and contaminating biological species in a composition.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc., including the same medium on which the program is presented.

In yet other embodiments, the instructions are not themselves present in the kit, but means for obtaining the instructions from a remote source, e.g., via the Internet, are provided. An example of this embodiment is a kit that includes a web address where the instructions can be viewed from or from where the instructions can be downloaded.

Still further, the kit may be one in which the instructions are obtained are downloaded from a remote source, as in the Internet or world wide web. Some form of access security or identification protocol may be used to limit access to those entitled to use the subject invention. As with the instructions, the means for obtaining the instructions and/or programming is generally recorded on a suitable recording medium.

### EXPERIMENTAL

### Example I

### Materials and Methods

DNA Extraction

A modified CTAB procedure (Doyle et al., 1997) was used to extract genomic DNA from fresh leaves of alfalfa (Medicago sativa), red clover (Trifolium pratense), woad (Isatis indigotica), European licorice (Glycyrrhiza glabra), Chinese licorice (Glycyrrhiza uralensis), or plant material contained in commercial alfalfa or red clover supplements of company A or B. Aliquots of DNA were run on a 1% agarose gel to check the quality of the DNA. A repair reaction (Leroy et al., 2000) was used on those samples that appeared degraded and failed to produce a PCR product.

### PCR Amplification

The polymerase chain reaction was carried out in a final volume of 20 ul with 1U Eppendorf Hotmaster Taq, 1×PCR buffer, 1.5 mM MgCl₂, 100 ng of each primer, 1 mM of each deoxynucleotide (dATP, dCTP, dTTP, dGTP) and 1 ul genomic DNA from fresh tissue or 4 ul repaired DNA. The primers used for amplification of the ITS region are as follows (described in Blattner et al., 1999): ITS-A (forward): 5′-GGAAGGAGAAGTCGTAACAAGG-3′; ITS-B (reverse): 5′-CTTTTCCTCCGCTTATTGATATG-3′; ITS-C (reverse): 5′-GCAATTCACACCAAGTATCGC-3′; and ITS-D (forward): 5′-CTCTCGGCAACGGATATCTCG-3′. The ITS-A and ITS-B universal primer pair produce a DNA fragment of 750 bp which includes the 3′-part of the 18S RNA, ITS-1, the 5.8S RNA, ITS-2, and the 5′-part of the 26S RNA. The PCR fragment generated using ITS-A and ITS-C is 360 bp long and includes the 3′-part of the 18S RNA and ITS-1. The ITS-D and ITS-B primer pair amplify the ITS-2 region between the 5.8S and 28S rRNA genes. The reverse primer in each amplification reaction was phosphorylated at the 5′ end to facilitate single strand DNA production from the resulting PCR product using lambda exonuclease (Schwieger and Tebbe 1998). PCR was conducted at 94° C. for 5 min, followed by 25 cycles of 30 s 94° C., 30 s 57° C., 1 min 30 S at 68° C., followed by a final extension step at 68° C. for 10 min.

### Genetic Profiling by Single Strand Conformational Polymorphism (SSCP)

The PCR products were purified using the Qiaquick PCR purification kit (Qiagen). Half the purified product was either heat denatured or used for digestion by Lambda exonuclease (Amersham Pharmacia Biotech) at 37° C. for 2 hours. The digested product was purified with the Qiagen Minielute kit and resuspended in 10 ul 1M Tris-HCl. Eight microliters of denaturing loading buffer (95% formamide, 10 mM NaOH, 0.25% bromophenol blue, 0.25% xylene cyanol) were added to each sample and were incubated at 95° C. for 3 min and snap cooled on ice. Samples were loaded on a 0.6×MDE (Mutational Detection Enhancement) gel (Cambrex, Rockland, Me.) using 0.4 mm spacers on a Vertical Gel Electrophoresis System (BRL, LifeTech, Inc., Maryland) and a 1×TBE (Sambrook and Russell, 2001) running buffer. Gels were run at room temperature at 7 mA and 90V for 14 hours. DNA was visualized by soaking the gels in 1 ug/mL ethidium bromide for 30 minutes.

### Extraction, Reamplification and Sequencing

Bands were cut out of ethidium bromide stained MDE gels and eluted at 37° C. for 2 hours in an elution buffer (0.5 M NH₄OAc, 10 mM MgOAc, 1 mM EDTA, 0.1% SDS). 1 ul eluent was used in PCR reactions using the same ITS-2 primer pairs used to generate the initial PCR product. The resulting PCR products were run on an agarose gel, purified using the Qiagen gel extraction kit, and sequenced using ABI Big Dye Terminator mix and automated sequencing with an ABI 3700 Capillary DNA Analyzer at the UCLA Sequencing Core Facility.

### Sequence Analysis

Sequences were compared to the database using NCBI BLAST.

### Results

### DNA Extraction and PCR Amplification

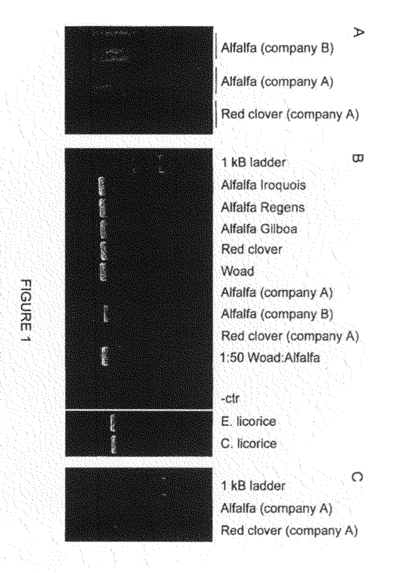

DNA was extracted from fresh leaves of Medicago sativa (alfalfa), Trifolium pratense (red clover), Glycyrrhiza uralensis (European licorice), Glycyrrhiza glabra (Chinese licorice) and Isatis indigotica (woad). Each of these species is reported to have medicinal properties. In addition, DNA was isolated from commercial products of red clover or alfalfa. Analysis of the genomic DNA on an agarose gel showed that DNA from each of the commercial products was degraded (FIG. 1A). Amplification of the ITS-2 region by PCR showed a single band for each of the species, which showed similar migration, indicating that differentiation based on standard agarose electrophoresis would not differentiate the species (FIG. 1B). A repair reaction was required for two of the commercial products before a PCR product could be amplified (FIG. 1C).

### SSCP can Differentiate Between Plant Species

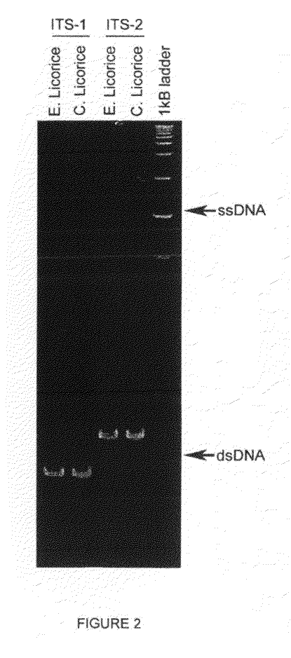

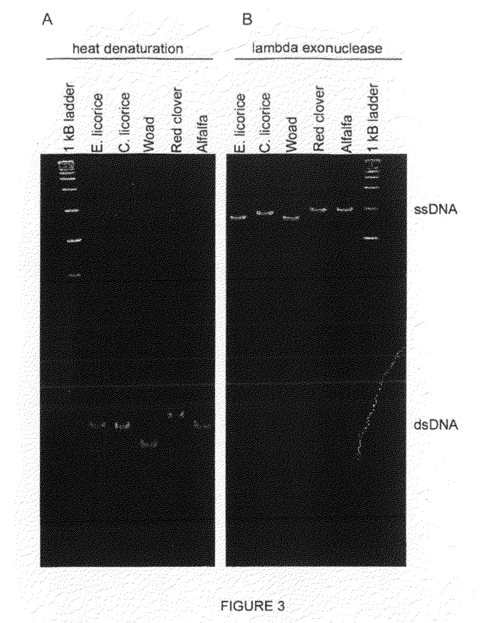

SSCP analysis of the ITS-1 region failed to differentiate between European and Chinese licorice (FIG. 2) with both ssDNA and dsDNA products showing similar migration on an acrylamide gel. In contrast, ssDNA products of the ITS-2 region migrated differently, with higher molecular weight bands appearing in Chinese licorice compared to European licorice. Analysis of the ITS-2 region for five different species showed that each could be differentiated based on the migration of double-stranded and single-stranded DNA (FIG. 3). A comparison of ssDNA products generated by heat denaturation (FIG. 3A) versus single strand digestion by exonuclease (FIG. 3B) showed that exonuclease digestion was more efficient at generating ssDNA products and produced fewer bands in Chinese licorice.

### SSCP can Distinguish Different Species in a Mixture

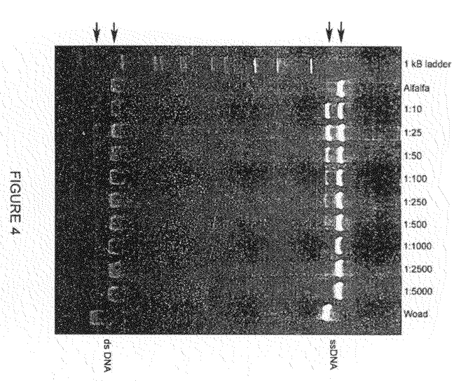

We prepared a simulated plant contamination event by mixing alfalfa and woad genomic DNA. We found that alfalfa and woad products amplified from the mix could clearly be differentiated based on their ssDNA or dsDNA (FIG. 4). To test the sensitivity of this technique for detecting contaminating plant material, we prepared a simulated contamination event using alfalfa DNA mixed with decreasing amounts of woad DNA. The woad contaminant could still be detected at a 1:5000 woad:alfalfa mixture (FIG. 4).

### Detection of Contaminating Plant Material in Commercial Products

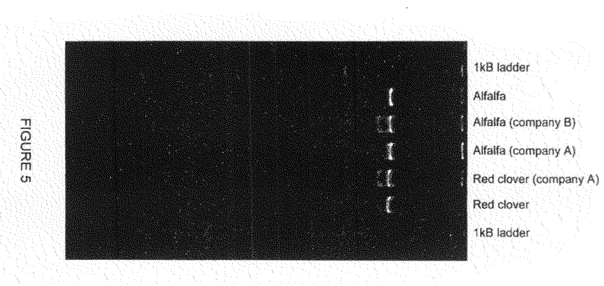

The SSCP technique was applied to commercial herbal supplements of alfalfa from two different manufacturers and to a red clover product. SSCP analysis of each supplement showed bands corresponding to that expected of red clover or alfalfa (FIG. 5). However, each supplement also revealed faster migrating bands that might be contaminants (FIG. 5). The identity of the single strand PCR products generated from each of the supplements were determined by extraction of each of the bands, PCR amplification and sequencing. Bands corresponding to the expected size were found to be alfalfa for the alfalfa products and red clover for the red clover product. The additional band in the alfalfa product from company A was found to be a species of Taraxacum (dandelion). The contaminant in the alfalfa supplement by the same company was identified as red clover. The contaminant in the red clover product from company B was identified as Trifolium polyssii.

### Discussion

Disclosed herein is a molecular method for discrimination and identification of the primary and contaminating plant species within a botanical mixture. The results analyzing plant mixtures show that SSCP combined with sequencing is a simple and effective technique for identifying species in mixed populations. SSCP analysis of the ITS2 region showed distinct variation of each of the five plant species we tested, including European and Chinese licorice, both members of the genus Glycyrrhiza. Furthermore, SSCP analysis combined with sequencing revealed contaminating plant material in commercial dietary supplements.

Molecular approaches to identifying the content of dietary supplements have been described before, but have not had the ability to readily detect multiple components in the product. Mihalov et al., (2000) used PCR combined with sequencing and DNA fingerprinting to identify ginseng in commercial samples. Through direct sequencing of a PCR product, they identified a product containing soybean. However, it was unclear whether multiple components, including ginseng, might also be present. RAPD analysis relies on the comparison to know molecular fingerprints, which makes it impossible to identify species for which a profile does not already exist.

SSCP was developed for the detection of mutations, specifically in human DNA (Orieta et al., 1989; Hayashi, 1991). However, it has become a useful tool in the study of communities and has successfully been used in population biology for the analysis of bacterial, fungal and even sponge populations (Schwieger and Tebbe, 1998; Jansa et al., 2002; Lopez et al., 2002). The application of SSCP to plants as disclosed herein demonstrates that this method can differentiate between plant species and therefore can be used as an assay to determine whether a sample contains multiple species. Traditional authentication of botanicals has relied on microscopic analysis or the assessment of marker compounds. The difficulty associated with these techniques is that the former requires the expertise of a taxonomist and the latter may be skewed by variation of marker content due to processing, tissue type, and environmental factors. SSCP applied first can demonstrate whether contaminants are present, and then the bands corresponding to possible contaminants can be further analyzed by sequencing.

The ITS region turned out to work well for differentiating plant species, as has been shown previously for microbes. The ITS region is commonly used for phylogenetic analysis of plants, resulting in a large collection of sequences in the database. This makes the ITS region extremely useful for the identification of botanicals. We found that for SSCP analysis, the ITS2 region gave better resolution than the ITS1 region, similar to what has been found for microbial species.

Analysis of commercial products of red clover and alfalfa using the disclosed methods found that each of the products were contaminated with other plant components. The alfalfa product from company A showed a Taraxacum contaminant. Taraxacum is a member of the dandelion family, and it is possible to speculate that it was growing in the field and harvested with the alfalfa. The same may be the case for the red clover contaminant identified in the alfalfa product from company B. However, as the same manufacturer produces both alfalfa and red clover supplements, it also seems feasible that the contamination occurred during the manufacturing process. The red clover supplement showed contamination by the closely related species Trifolium polysii. This particular species is considered endangered, and the lack of an exact match makes it possible that the contaminant came from a different Trifolium species that is not yet present in the database. Another possibility is that cross species hybridization has occurred.

The method described in this invention is applicable to the identification of primary and contaminating biological species in botanical products. SSCP combined with sequencing allows for the positive identification of specific species, even those that are very closely related. This method is therefore useful in the assessment of the quality of commercial botanical products including foods, dietary supplements and therapeutics.

What is claimed is:

1. A method for determining the identity of biological species in a botanical composition said method comprising:
(a) isolating nucleic acid from said botanical composition;
(b) generating a product from a genomic identification region of said isolated nucleic acid using PCR amplification;
(c) generating single-stranded DNA from said PCR-amplified product;
(d) identifying and isolating said individual species of said single stranded DNA product; and
(e) obtaining the nucleotide sequence of each of said species of said single stranded DNA product and comparing it to known sequences to identify the primary biological composition of said botanical composition.

2. The method according to claim 1, wherein said isolated nucleic acid from said botanical is repaired prior to said PCR-amplification step.

3. The method according to claim 1, wherein said genomic identification region is an internal transcribed spacer (ITS) region of the ribosomal RNA gene.

4. The method according to claim 1, wherein generation of single stranded DNA is done using lambda exonuclease.

5. The method according to claim 1, wherein the identification and isolation of said single-stranded DNA species is done using SSCP.

6. The method according to claim 1, wherein prior to obtaining said sequence of said single-stranded DNA product (e) said single stranded DNA product is PCR-amplified.
